(19) United States
(12) Patent Application Publication
(10) Pub. No.: US 2025/0375430 A1
(43) Pub. Date: Dec. 11, 2025

(54) USE OF DIHYDROBERBERINE TO PROMOTE RECRUITMENT AND ACTIVATION OF BROWN ADIPOSE TISSUES

(71) Applicant: NANJING NUTRABUILDING BIO-TECH CO., LTD., Nanjing (CN)

(72) Inventors: Ronghua YI, Nanjing (CN); Kylin LIAO, Nanjing (CN); Shawn WELLS, Frisco, TX (US)

(21) Appl. No.: 19/298,469

(22) Filed: Aug. 13, 2025

Related U.S. Application Data
(63) Continuation of application No. PCT/CN2024/075782, filed on Feb. 4, 2024.

(30) Foreign Application Priority Data
Feb. 14, 2023 (WO) PCT/CN2023/075793

(51) Int. Cl. A61K 31/4375; A61P 3/04; A61P 3/06; A61P 3/10

(52) U.S. Cl. CPC A61K 31/4375; A61P 3/04; A61P 3/06; A61P 3/10

(57) ABSTRACT

The present invention provides novel methods for promoting recruitment and activation of brown adipose tissues (BAT) or arousing browning of white adipose tissues (WAT) in a mammal, comprising administration to the mammal a composition comprising an effective amount of dihydroberberine or a pharmaceutically acceptable salt, acid, ester, polymer, analog or derivative thereof. The present invention also provides a composition comprising an effective amount of dihydroberberine or a pharmaceutically acceptable salt, acid, ester, polymer, analog or derivative thereof, for promoting recruitment and activation of brown adipose tissues (BAT) or arousing browning of white adipose tissues (WAT) in a mammal.

### CROSS REFERENCE TO RELATED APPLICATION

This application is a continuation application of International Patent Application No. PCT/CN2024/075782, filed on Feb. 4, 2024, which claims the priority of the International Patent Application No. PCT/CN2023/075793, filed on Feb. 14, 2023, the contents of all of which are incorporated herein by reference in their entirety.

### BACKGROUND OF THE INVENTION

### Technical Field

The present invention generally relates to compositions and methods for promoting recruitment and activation of brown adipose tissues (BAT) or arousing browning of white adipose tissues (WAT) in a mammal.

### Background Technology

Mammals possess two specialized types of adipose tissues that serve opposite functions, white and brown adipose tissues (WAT and BAT for short respectively). WAT, which is characterized by large unilocular lipid-droplets-containing white adipocytes, is an active endocrine organ that regulates diverse activities, such as insulin sensitivity. BAT and WAT are morphologically and functionally different tissues, and their developmental patterns are quite distinct. BAT is histologically distinct from WAT, it composes of multilocular lipid droplets and large numbers of mitochondria that contain mitochondrial uncoupling protein-1 (UCP1). BAT is specialized to combust energy by a process termed non-shivering thermogenesis mediated by mitochondrial UCP1. In addition, BAT plays an active role in energy expenditure by oxidizing fatty acids that are produced by triglyceride hydrolysis to generate heat. Moreover, BAT also could dissipate chemical energy to protect against hypothermia and adipocyte dysfunctions through non-shivering thermogenesis.

Two types of thermogenic adipocytes exist: classical brown adipocytes that are abundant in BAT with the high level of UCP1, and beige/brite adipocytes that are abundant in WAT with the low level of UCP1 under basal conditions. So, it is necessary to trigger the “browning of white fat”. However, beige/brite adipocytes have potential to express the comparable level of UCP1 to brown adipocytes in response to stimuli such as cold temperatures or β3-adrenergic receptor agonists. Several crucial transcriptional factors involved in brown and beige/brite adipocyte development have recently been identified. Among them, PRD1-BFI-RIZI homologous domain containing 16 (PRDM16) and UCP1 act as the master regulators of brown/beige adipogenesis through their interactions with transcriptional factors, such as peroxisome proliferator-activated receptor-γ co-activator 1α (PGC-1α), and C-terminal-binding protein 1, etc.

The activity of human BAT is negatively correlated with age, BMI, fat mass, and fasting blood glucose (FBG), and positively correlated with resting metabolic rate. Besides, it has been demonstrated that BAT in human adults has many considerable functions, such as generating heat from fat, increasing energy expenditure, and improving glucose metabolism and insulin sensitivity. Thus, pharmacological or nutritional strategies targeting BAT provide an appealing option for the metabolic disorders field, as little medicine or dietary supplement have been identified to effectively and safely recruit or activate BAT in humans.

Until today, the traditional Chinese medicine, called berberine (BBR), a natural plant alkaloid originally isolated from extracts of Berberis aristata and Coptis chinensis (Huanglian), is widely used for anti-diarrhoea, and has been found to have a variety of metabolic benefits in recent years. Additionally, BBR has been demonstrated to promote brown adipocyte differentiation and BAT thermogenesis by increasing the transcription of PRDM16 driven via AMPK activation and PGC-la recruitment. Similarly, another study demonstrated that BBR could reduce weight gain, promote the thermogenic program in BAT, and increase energy expenditure and adaptive thermogenesis in obese db/db mice.

Notably, dihydroberberine (DHB), a hydrogenated derivative of BBR, also has these similar benefits and it works better according to our experiment. Furthermore, in vitro, some scientists found that the intestinal absorption rate of DHB was five times higher than BBR. These findings were then tested in practice on mice, and the researchers found that DHB was better absorbed than BBR, including in the mice lacking the necessary intestinal bacteria needed to break down BBR. That is to say, DHB is a far superior ingredient and has more bioavailable than BBR.

Taken together, in this invention, we found that DHB could be considered as a promising and attractive ingredient to promote recruitment and activation of BAT.

### SUMMARY OF THE PRESENT INVENTION

In a first aspect, the present invention provides a method for promoting recruitment and activation of brown adipose tissues (BAT) or arousing browning of white adipose tissues (WAT) in a mammal, the method comprising administration to the mammal a composition comprising an effective amount of dihydroberberine or a pharmaceutically acceptable salt, acid, ester, polymer, analog or derivative thereof.

In some embodiments, the promoting recruitment and activation of brown adipose tissues (BAT) or arousing browning of white adipose tissues (WAT) is achieved by inducing brown/brite-typical gene expression, like UCP1 and PGC-1α; or increasing the transcription of PRDM16 and UCP1 via AMPK activation and PGC-1α recruitment.

In some embodiments, the method is used for improving biomarkers related to lipid mobilization; enhancing or inducing BAT thermogenesis and energy expenditure; reducing body weight, decreasing body fat percentage; improving disordered metabolic profiles.

In some embodiments, the improving biomarkers related to lipid mobilization comprises decreasing triglyceride level, total cholesterol level, low-density lipoprotein level, very low-density lipoprotein level: the enhancing or inducing BAT thermogenesis and energy expenditure comprises generating heat from fat, increasing energy expenditure; the improving disordered metabolic profiles comprises improving glucose metabolism and insulin sensitivity, or reducing blood glucose and insulin levels.

In some embodiments, the mammal is human or cattle or pet. In some embodiments, the mammal is human.

In some embodiments, the composition is prepared in a form of food, drink, nutritional supplement, cosmetic product, or pharmaceutical composition.

In some embodiments, the administration is through various routes selected from oral administration, intravenous injection, intramuscular injection, intraperitoneal injection, topical application, or sublingual application.

In some embodiments, the dihydroberberine or a pharmaceutically acceptable salt, acid, ester, polymer, analog or derivative thereof is administered at a daily dose of 10 mg-2000 mg. In some embodiments, the dihydroberberine or a pharmaceutically acceptable salt, acid, ester, polymer, analog or derivative thereof is administrated at a daily dose of 20-1500 mg, 40-1000 mg, 60-800 mg, 80-600 mg, or 100-400 mg. In some embodiments, the administration is at least once a day or more times a day. In some embodiments, the composition is administered in divided doses or a single dose.

In some embodiments, the dihydroberberine or a pharmaceutically acceptable salt, acid, ester, polymer, analog or derivative thereof is formulated in solutions, liquid suspensions, parenteral solutions, injections, tablets, pills, granules, powders, films, (micro)capsules, aerosols, tonics, syrups, beverages, nourishments, snacks, bars, gums, sugars, a facial mask composition, a functionalized cream composition, a functionalized essence, a skin care composition, a make-up composition or a functionalized food composition.

In a second aspect, the present invention provides a composition comprising an effective amount of dihydroberberine or a pharmaceutically acceptable salt, acid, ester, polymer, analog or derivative thereof, for promoting recruitment and activation of brown adipose tissues (BAT) or arousing browning of white adipose tissues (WAT) in a mammal.

In some embodiments, the promoting recruitment and activation of brown adipose tissues (BAT) or arousing browning of white adipose tissues (WAT) is achieved by inducing brown/brite-typical gene expression, like UCP1 and PGC-1α; or increasing the transcription of PRDM16 and UCP1 via AMPK activation and PGC-1α recruitment.

In some embodiments, the composition is used for improving biomarkers related to lipid mobilization; enhancing or inducing BAT thermogenesis and energy expenditure; reducing body weight, decreasing body fat percentage; improving disordered metabolic profiles.

In some embodiments, the improving biomarkers related to lipid mobilization comprises decreasing triglyceride level, total cholesterol level, low-density lipoprotein level, very low-density lipoprotein level; the enhancing or inducing BAT thermogenesis and energy expenditure comprises generating heat from fat, increasing energy expenditure; the improving disordered metabolic profiles comprises improving glucose metabolism and insulin sensitivity, or reducing blood glucose and insulin levels.

In some embodiments, the mammal is human or cattle or pet. In some embodiments, the mammal is human.

In some embodiments, the composition is prepared in a form of food, drink, nutritional supplement, cosmetic product, or pharmaceutical composition.

In some embodiments, the dihydroberberine or a pharmaceutically acceptable salt, acid, ester, polymer, analog or derivative thereof is administered at a daily dose of 10 mg-2000 mg. In some embodiments, the dihydroberberine or a pharmaceutically acceptable salt, acid, ester, polymer, analog or derivative thereof is administrated at a daily dose of 20-1500 mg, 40-1000 mg, 60-800 mg, 80-600 mg, or 100-400 mg. In some embodiments, the administration is at least once a day or more times a day. In some embodiments, the composition is administered in divided doses or a single dose.

In some embodiments, the dihydroberberine or a pharmaceutically acceptable salt, acid, ester, polymer, analog or derivative thereof is formulated in solutions, liquid suspensions, parenteral solutions, injections, tablets, pills, granules, powders, films, (micro)capsules, aerosols, tonics, syrups, beverages, nourishments, snacks, bars, gums, sugars, a facial mask composition, a functionalized cream composition, a functionalized essence, a skin care composition, a make-up composition or a functionalized food composition.

In a third aspect, the present invention provides use of a composition for preparing food, drink, nutritional supplement, cosmetic product, or pharmaceutical composition for promoting recruitment and activation of brown adipose tissues (BAT) or arousing browning of white adipose tissues (WAT) in a mammal, the composition comprises an effective amount of dihydroberberine or a pharmaceutically acceptable salt, acid, ester, polymer, analog or derivative thereof.

In some embodiments, the promoting recruitment and activation of brown adipose tissues (BAT) or arousing browning of white adipose tissues (WAT) is achieved by inducing brown/brite-typical gene expression, like UCP1 and PGC-1α, or increasing the transcription of PRDM16 and UCP1 via AMPK activation and PGC-1α recruitment.

In some embodiments, the composition is used for improving biomarkers related to lipid mobilization; enhancing or inducing BAT thermogenesis and energy expenditure; reducing body weight, decreasing body fat percentage; improving disordered metabolic profiles.

In some embodiments, the improving biomarkers related to lipid mobilization comprises decreasing triglyceride level, total cholesterol level, low-density lipoprotein level, very low-density lipoprotein level; the enhancing or inducing BAT thermogenesis and energy expenditure comprises generating heat from fat, increasing energy expenditure; the improving disordered metabolic profiles comprises improving glucose metabolism and insulin sensitivity, or reducing blood glucose and insulin levels.

In some embodiments, the dihydroberberine or a pharmaceutically acceptable salt, acid, ester, polymer, analog or derivative thereof is administered at a daily dose of 10 mg-2000 mg. In some embodiments, the dihydroberberine or a pharmaceutically acceptable salt, acid, ester, polymer, analog or derivative thereof is administrated at a daily dose of 20-1500 mg, 40-1000 mg, 60-800 mg, 80-600 mg, or 100-400 mg. In some embodiments, the administration is at least once a day or more times a day. In some embodiments, the composition is administered in divided doses or a single dose.

In some embodiments, the dihydroberberine or a pharmaceutically acceptable salt, acid, ester, polymer, analog or derivative thereof is formulated in solutions, liquid suspensions, parenteral solutions, injections, tablets, pills, granules, powders, films, (micro)capsules, aerosols, tonics, syrups, beverages, nourishments, snacks, bars, gums, sugars, a facial mask composition, a functionalized cream composition, a functionalized essence, a skin care composition, a make-up composition or a functionalized food composition.

These and other features, aspects, and advantages of the present invention will become better understood with reference to the following description and appended claims.

### BRIEF DESCRIPTION OF THE DRAWINGS

FIG. 1 is a graph of TG results in all groups.

FIG. 2 is a graph of TC results in all groups.

FIG. 3 is a graph of LDL results in all groups.

FIG. 4 is a graph of VLDL results in all groups.

FIG. 5 is a graph of i.p. glucose tolerance results in all groups.

FIG. 6 is a graph of insulin tolerance results in all groups.

FIG. 7 is a graph of body weight at every supplementation week (week 9 to week 14) in all groups.

FIG. 8 is a graph of fat mass percentage at 9^{th} and 14^{th} week in all groups.

FIG. 9 is a graph of thermogenic gene expression (UCP1 and PGC-1α) levels in all groups.

FIG. 10 is a graph of expression levels of UCP1, PRDM16 and PGC-1α in all groups.

FIG. 11 is a graph of average oxygen consumption (VO₂) over a 24 h period in all groups.

FIG. 12 is a graph of carbon dioxide release (VCO₂) over a 24 h period in all groups.

FIG. 13 is a graph of energy expenditure over a 24 h period in all groups.

FIG. 14 is a graph of total ¹⁸F-FDG uptake in BAT in all groups. FIG. 15 is a graph of rectal temperature at 9^{th} and 14^{th} week in all groups.

### DETAILED DESCRIPTION OF THE INVENTION

In the Summary Section above and the Detailed Description Section, and the claims below, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” and their conjugations, refer to a situation wherein said terms are used in their non-limiting sense to mean that items following the word are included, but items not specifically mentioned are not excluded. It also encompasses the more limiting verb ‘to consist essentially of’ and ‘to consist of’.

As used herein, the term “effective amount” refers to the amount required to achieve the effect as taught herein. The specific effective dose level for any particular subject will depend upon a variety of factors including the conditions being treated and the severity of the conditions; the specific composition employed; the age, body weight, general health, sex and diet of the subject; the time of administration, route of administration, and rate of excretion of dihydroberberine or its analog or its derivatives employed; the duration of the treatment; and like factors well known in the medical arts. For example, it is well known within the skill of the art to start doses of the compound at levels lower than those required to achieve the desired effect and to gradually increase the dosage until the desired effect is achieved.

One of skill in the art recognizes that an amount may be considered “effective” even if the condition is not totally eradicated or prevented, but it or its symptoms and/or effects are improved or alleviated partially in the subject.

As used herein, the term “pharmaceutically acceptable” means pharmaceutically, physiologically, alimentarily, and/or nutritionally acceptable, and refers to those compositions or combinations of agents, materials, or compositions, and/or their dosage forms, which are within the scope of sound medical judgment, suitable for use in contact with the tissues of human beings and animals without excessive toxicity, irritation, allergic response, or other problem or complication, commensurate with a reasonable benefit/risk ratio.

As used herein, the term “mammal” or “subject” may be used interchangeably to refer to any animal to which the presently disclosed methods and compositions may be applied or administered. The animal may have an illness or other disease, but the animal does not need to be sick to benefit from the presently disclosed methods and compositions. As such any animal may apply the disclosed combinations, compositions or kits, or be a recipient of the disclosed methods. “Mammal” includes, without limitation, mice, rats, rabbits, guinea pigs, dogs, cats, sheep, goats, cows, horses, primates, such as monkeys, chimpanzees, and apes, and, in particular, humans. Although the animal subject is preferably a human, the methods and compositions of the invention have application in veterinary medicine.

The dosage of dihydroberberine or a pharmaceutically acceptable salt, acid, ester, polymer, analog or derivative thereof and/or composition comprising the same may range broadly, depending upon the desired effects and the indication. The daily dosage regimen for an adult human patient may be, for example, an oral dose of between 0.01 mg and 3000 mg of dihydroberberine or its analog or derivative, preferably between 1 mg and 700 mg, e.g. 5 to 200 mg, or between about 0.1 mg and about 1,000 mg of dihydroberberine or its analog or derivative per kg of body weight of the subject. The dosage may be a single one or a series of two or more given in the course of one or more days, as is needed by the subject. In some embodiments, the compounds are administered for a period of time, for example for a week or more, or for months or years.

As used herein, the term “administration” refers to the process of delivering a disclosed composition or active ingredient to a subject. The compositions of the invention can be administered in a variety of ways, including orally, intragastrically, and parenterally (e.g., intravenous and intraarterial as well as other suitable parenteral routes), and the like.

As used herein, a “parenteral solution” refers to a solution that can be administered elsewhere in the body than the mouth and alimentary canal. It is not delivered via the intestinal tract. For example, parenteral solution can be delivered intravenously.

As used herein, a “tonic” refers to a medicinal substance taken to give a feeling of vigor or well-being.

As used herein, a “syrup” refers to a thick sticky liquid derived from a sugar-rich plant. for example, sugar cane, corn, and maple.

Multiple techniques of administering a composition exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections.

“Intraperitoneal” as used here means within or administered through the peritoneum. The peritoneum is a thin, transparent membrane that lines the walls of the abdominal (peritoneal) cavity and contains/encloses the abdominal organs such as the stomach and intestines.

As used herein, “sublingual” refers to situated or applied under the tongue.

A “functionalized cream composition” includes a cream composition that has a potentially positive effect on health beyond basic nutrition.

An “essence” includes an extract or concentrate obtained from a particular plant or other matter and used for flavoring or scent A “functionalized essence” includes an essence that has a potentially positive effect on health beyond basic nutrition.

A “functionalized food composition” includes a food composition that has a potentially positive effect on health beyond basic nutrition.

Any titles or subheadings used herein are for organization purposes and should not be used to limit the scope of embodiments disclosed herein.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for.

All publications and patent applications cited in this specification are herein incorporated by reference as if each individual publication or patent application were specifically and individually indicated to be incorporated by reference.

### Examples

Six-week-old male C57BL/6J mice were housed in a temperature-and relative humidity-controlled room (22±2° C., 55±5%) with a 12 h light-dark cycle and free access to food and water. For chronic anti-obesity studies, all mice were fed the high-fat diet (HFD, 60% calories from fat, 20% calories from protein, 20% calories from carbohydrate) for 8 weeks.

At 14 weeks of age, all mice were randomly divided into 3 groups of 10 animals: (1) non-supplement group (Con Food only, Group 1); (2) DHB supplement group (DHB+Food. 50 mg/kg, Group 2); (3) DHB supplement group (DHB+Food, 100 mg/kg, Group 3).

After an eight-week induction, the mice in group I were intraperitoneal injection (i.p.) administrated with normal saline between 9:30 and 11:30 am, and food and water were given ad libitum.

The mice in group 2 and group 3 were intraperitoneal injection (i.p.) administrated with DHB in normal saline solution daily for 6 weeks between 9:30 and 11:30 am, and food and water were given ad libitum.

The total experimental time is 14 weeks, including two periods: an eight-week induction period and a subsequent six-week supplement period.

At the end of the study, all mice were anesthetized with chloral hydrate, all relevant tissues like inguinal and perirenal adipose were dissected, weighed, immediately frozen in liquid nitrogen, and stored at −80° C.

### EXAMPLE 1. DHB improves Biomarkers Related to Lipid Mobilization in Mammals

Plasma total cholesterol (TC), triglyceride (TG), low-density lipoprotein (LDL), and very low-density lipoprotein (VLDL) were measured using corresponding kits according to the manufacturers' instructions.

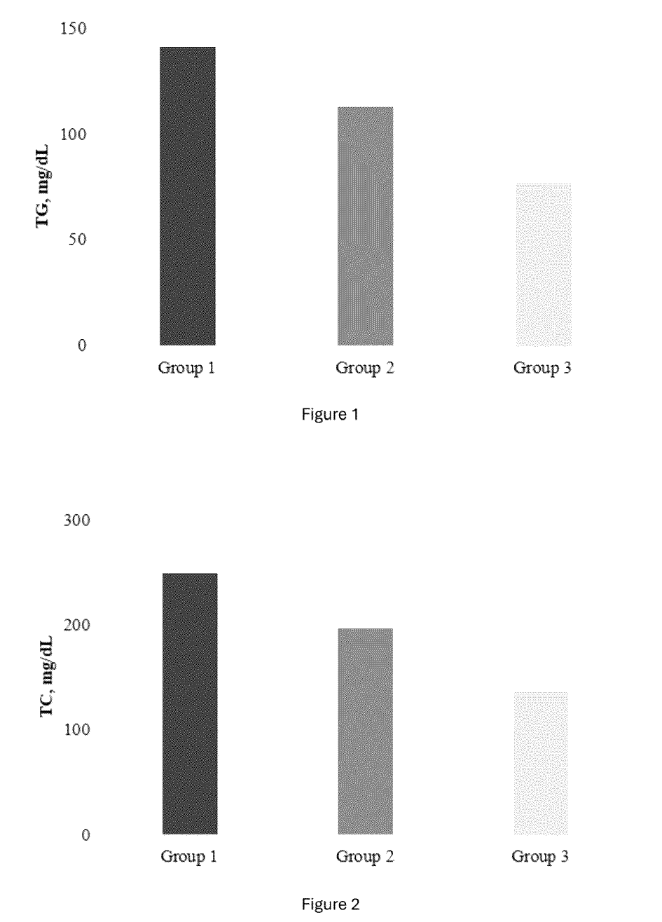

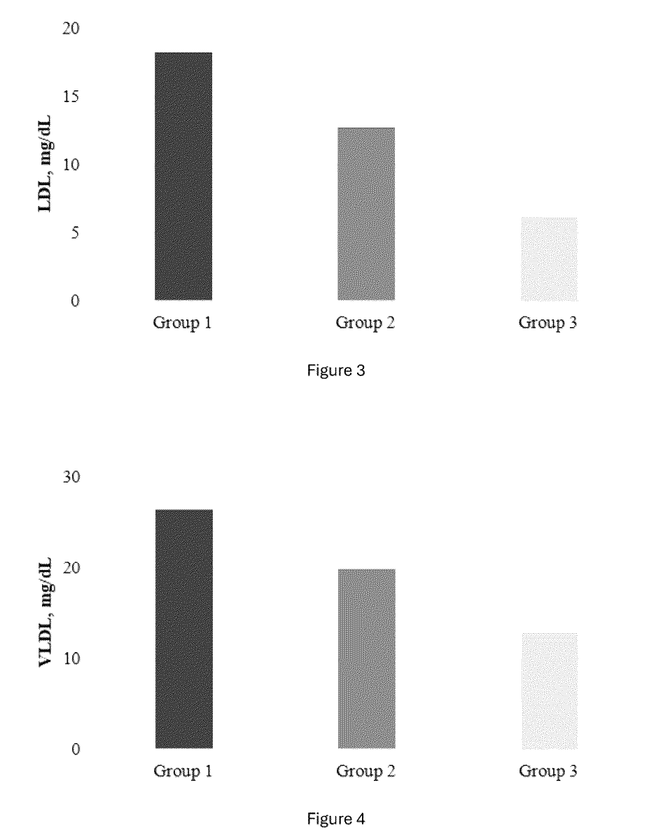

FIG. 1 is a graph of TG results in all groups. FIG. 2 is a graph of TC results in all groups. FIG. 3 is a graph of LDL results in all groups. FIG. 4 is a graph of VLDL results in all groups. In FIGS. 1-4, it is observed that DHB supplementation could have lower levels of TC, TG, LDL, VLDL, relative to mice without receiving DHB supplementation, indicating that supplementing DHB can achieve the beneficial effects on improving biomarkers related to lipid mobilization than without DHB supplementation. Additionally, the higher dose of DHB can have greater effects on improving biomarkers related to lipid mobilization than the lower dose of DHB. This effect persists for as long as the test protocol is continued.

### EXAMPLE 2. DHB Reduces the Body Weight, Decreases Body Fat Percentage, and Improves Disordered Metabolic Profiles, Like Reducing Blood Glucose and Insulin Levels in Mammals

A glucose tolerance test (GTT) was performed in the obese mice overnight by using a glucose analyzer in the 4th week of supplementation. The glucose concentrations were measured in blood collected by venous bleeding from the tail vein immediately at 0, 15, 30, 60 and 120 min after a bolus i.p. injection of glucose at 0.75 g/kg.

An insulin tolerance test (ITT) was performed by using a glucose analyzer in the 6-h fasting mice in the 5^{th} week of the supplementation. The glucose concentrations were measured by venous bleeding at 0, 15, 30, 60 and 120 min after an i.p. injection of human insulin at 1 U/kg.

The body weight and fat mass of all mice were performed by the nuclear magnetic resonance body composition analyzer by Nuclear Magnetic Resonance Body Composition Analyzer in accordance with manufacturer's instruction. The body weight and fat mass were tested weekly (9-14 weeks) and at 9^{th} and 14^{th} week before supplementation and feeding, respectively.

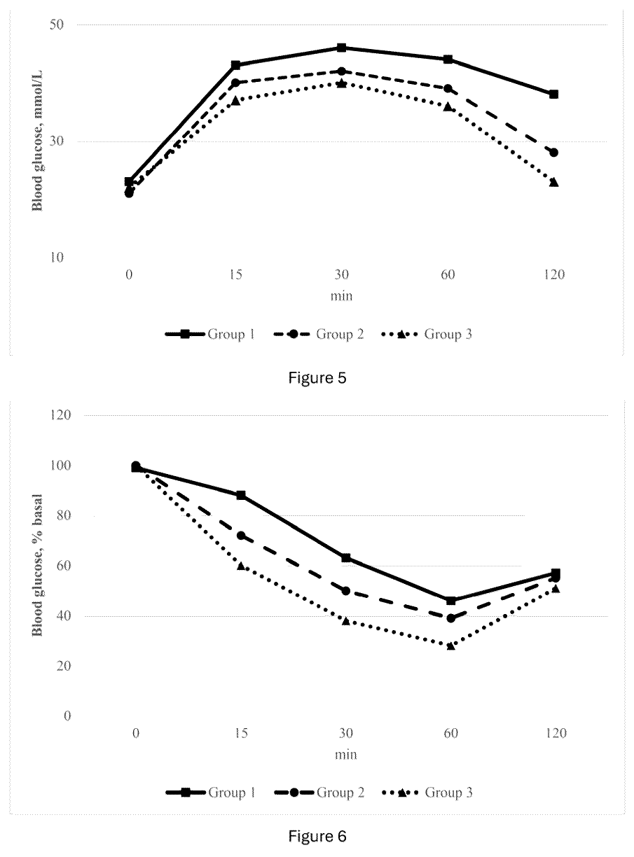

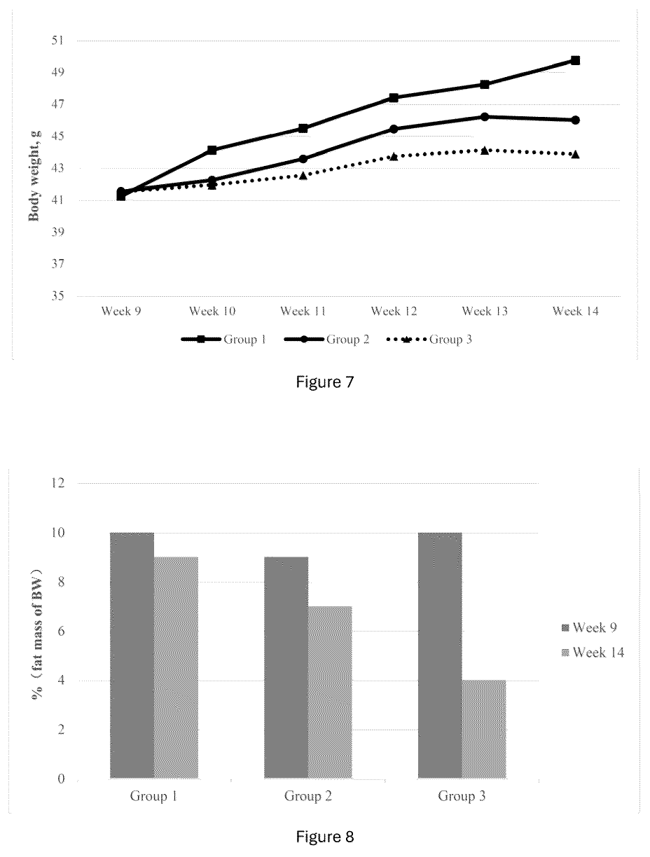

FIG. 5 is a graph of i.p. glucose tolerance results in all groups. FIG. 6 is a graph of insulin tolerance results in all groups. FIG. 7 is a graph of body weight at every supplementation week (week 9 to week 14) in all groups. FIG. 8 is a graph of fat mass percentage at 9^{th} and 14^{th} week in all groups. In FIG. 5, it is observed that mice in groups receiving DHB supplementation, especially higher dose, had a better tolerance to a glucose load which was particularly prominent in almost all time points of an i.p. glucose tolerance test. Combined with another insulin tolerance (FIG. 6), DHB ameliorated glucose tolerance, and the higher-dose group had better result. In addition, the group 2 and 3 were obviously protected from weight gain during 6 weeks of treatment (FIG. 7). Significantly, mice in group 3 inhibited fat accumulation better (FIG. 8). To sum up, these results demonstrated that DHB reduced the body weight decreases body fat percentage and improved disordered metabolic profiles, like reducing blood glucose and insulin levels relative to mice without receiving DHB supplementation, indicating that supplementing DHB can achieve the beneficial effects on metabolic health than without DHB supplementation. Additionally, the higher dose of DHB can have greater effects on metabolic health than the lower dose of DHB. This effect persists for as long as the test protocol is continued.

### EXAMPLE 3. DHB Arouses Browning of WAT, Like Inguinal WAT, by Inducing Brown/Brite-Typical Gene Expression, Like UCP1 and PGC-1α in Mammals

The mRNA levels of brown fat-enriched genes such as UCP1 and PGC-1α from adipose tissues (like inguinal WAT) were determined by real-time PCR analysis.

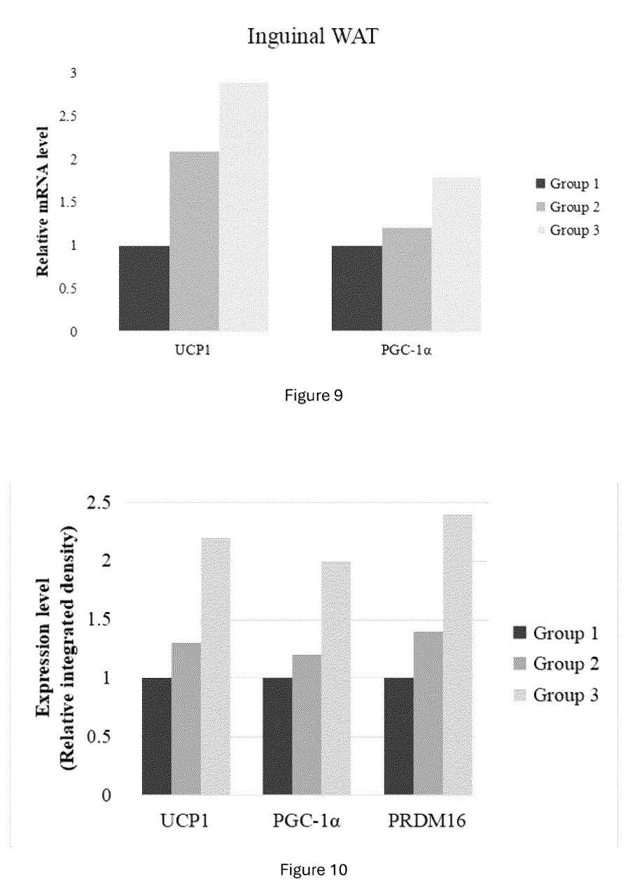

FIG. 9 is a graph of thermogenic gene expression (UCP1 and PGC-1α) levels in all groups. As shown in FIG. 9, DHB supplementation could increase the higher levels of UCP1 and PGC-1α by real-time PCR, relative to mice without receiving DHB supplementation, indicating that supplementing DHB can achieve the beneficial effects on the “browning of white fat” than without DHB supplementation. Additionally, the higher dose of DHB can have greater effects on the “browning of white fat” than the lower dose of DHB. This effect persists for as long as the test protocol is continued.

### EXAMPLE 4. DHB Enhances or Induces BAT Thermogenesis and Energy Expenditure by Increasing the Transcription of Brown Fat Marker PRDM16 and UCP1 Via AMPK Activation and Adipogenic Factor PGC-1α Recruitment in Mammals

Mice were allowed to fast overnight and were lightly anesthetized with isoflurane followed by a tail vein injection of ¹⁸F-FDG (150 mCi). Sixty mins after the injection of the radiotracer, the mice were subjected to PET/CT analysis. Inveon Acquisition Workplace (IAW) 1.5.0.28 was used for the scanning process. A 10 min CT X-ray for attenuation correction was scanned with a power of 80 kv and 500 mA and an exposure time of 1,100 ms before the PET scan. Ten-minute static PET scans were acquired, and the images were reconstructed by an OSEM3D algorithm followed by Maximization/Maximum a Posteriori (MAP) or FastMAP provided by IAW. The three-dimensional regions of interest (ROls) were drawn over the guided CT images, and the tracer uptake was measured using the software of Inveon Research Workplace (IRW) 3.0. Individual quantification of the ¹⁸F-FDG uptake in each of the ROIs was calculated. The data for the accumulation of ¹⁸F-FDG on microPET images were expressed as the standard uptake values, which were determined by dividing the relevant ROI concentration by the ratio of the injected activity to the body weight. The total accumulated amount of ¹⁸F-FDG in a ROI on microPET images was calculated by the following formula.

$$FDG uptake ( kBq ) = \frac{SUV mean ( \frac{g}{mL} ) \times radiotracer activity ( μ C i ) \times ( \frac{kBq}{μ Ci} ) \times ROI volume ( mL )}{Body weight ( g )}$$

A key factor for controlling energy homoeostasis is the balance between caloric intake and energy expenditure. We analyzed the actions of DHB on energy expenditure using comprehensive lab animal monitoring system (CLAMS). Metabolic analysis was conducted between 8:30 am and 7:30 pm at the last week of experiment to identify the maximal and mean standardized uptake value (SUV) of BAT. O₂ consumption, CO₂ production, and locomotor activity were measured using a 16-chamber indirect calorimeter. The mice were acclimated to the system for 20-24 h, and measurement of VO₂ and VCO₂ was performed during the next 24 h. The mice were maintained at 24° C. under a 12 h light/dark cycle. Food and water were available ad libitum. Voluntary activity was derived from the x-axis beam breaks monitored every 15 min. Heat production (energy expenditure) was calculated using the following equation:

$$Heat = [ 3.815 + 1.232 ( {V}_{CO 2} / {V}_{CO 2} ) ] \times {V}_{O 2} \times body weight ,$$

where heat is measured in kcal h⁻¹, VO₂ is measured in litres kg⁻¹ h⁻¹ and body weight is measured in kg.

Mice were placed in the cold room (at 4-8° C. without food but with free access to water), and measured rectal temperature in 20 min intervals between 9:30 and 11:30 am using a rectal probe attached to a digital thermometer in the 9^{th} week and 14^{th} week.

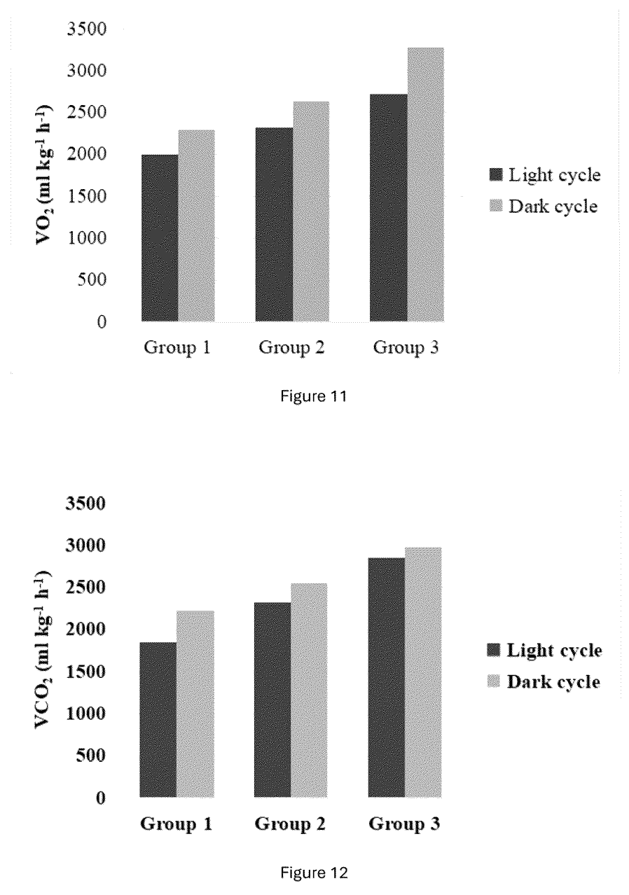

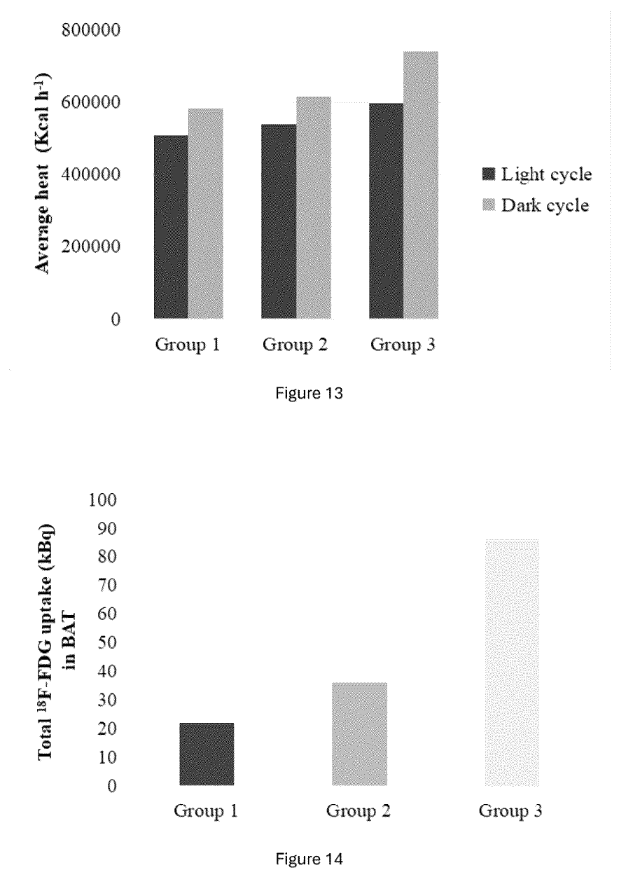

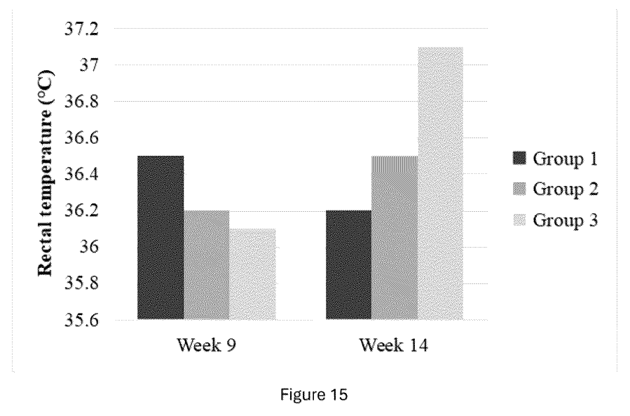

FIG. 10 is a graph of expression levels of UCP1, PRDM16 and PGC-1α in all groups. FIG. 11 is a graph of average oxygen consumption (VO₂) over a 24 h period in all groups. FIG. 12 is a graph of carbon dioxide release (VCO₂) over a 24 h period in all groups. FIG. 13 is a graph of energy expenditure over a 24 h period in all groups. FIG. 14 is a graph of total ¹⁸F-FDG uptake in BAT in all groups. FIG. 15 is a graph of rectal temperature at 9^{th} and 14^{th} week in all groups. It is manifested in FIGS. 10-15 that DHB-supplemented mice showed markedly higher oxygen consumption and carbon dioxide production rates through a 12-h light/dark cycle, higher rectal temperature, increase the accumulated amount of ¹⁸F-FDG and increase the expression of brown fat marker PRDM16 and UCP1 as well as the adipogenic factor PGC-1α, relative to mice without receiving DHB supplementation, indicating that supplementing DHB can achieve the beneficial effects on BAT thermogenesis and energy expenditure by increasing the transcription of brown fat marker PRDM16 and UCP1 via AMPK activation and adipogenic factor PGC-1α recruitment. Additionally, the higher dose of DHB can have greater effects on BAT thermogenesis and energy expenditure than the lower dose of DHB. This effect persists for as long as the test protocol is continued.

Although specific embodiments and examples of this invention have been illustrated herein, it will be appreciated by those skilled in the art that any modifications and variations can be made without departing from the spirit of the invention. The examples and illustrations above are not intended to limit the scope of this invention. Any combination of embodiments of this invention, along with any obvious their extension or analogs, are within the scope of this invention. Further, it is intended that this invention encompass any arrangement, which is calculated to achieve that same purpose, and all such variations and modifications as fall within the scope of the appended claims.

What is claimed is:

1. A method for promoting recruitment and activation of brown adipose tissues (BAT) or arousing browning of white adipose tissues (WAT) in a mammal, the method comprising administration to the mammal a composition comprising an effective amount of dihydroberberine or a pharmaceutically acceptable salt, acid, ester, polymer, analog or derivative thereof.

2. The method of claim 1, wherein the promoting recruitment and activation of brown adipose tissues (BAT) or arousing browning of white adipose tissues (WAT) is achieved by inducing brown/brite-typical gene expression, like UCP1 and PGC-1α; or increasing the transcription of PRDM16 and UCP1 via AMPK activation and PGC-1α recruitment.

3. The method of claim 1, wherein the method is used for improving biomarkers related to lipid mobilization; enhancing or inducing BAT thermogenesis and energy expenditure; reducing body weight, decreasing body fat percentage; improving disordered metabolic profiles.

4. The method of claim 3, wherein the improving biomarkers related to lipid mobilization comprises decreasing triglyceride level, total cholesterol level, low-density lipoprotein level, very low-density lipoprotein level; the enhancing or inducing BAT thermogenesis and energy expenditure comprises generating heat from fat, increasing energy expenditure; the improving disordered metabolic profiles comprises improving glucose metabolism and insulin sensitivity, or reducing blood glucose and insulin levels.

5. The method of claim 1, wherein the mammal is human or cattle or pet.

6. The method of claim 1, wherein the composition is prepared in a form of food, drink, nutritional supplement, cosmetic product, or pharmaceutical composition.

7. The method of claim 1, wherein the administration is through various routes selected from oral administration, intravenous injection, intramuscular injection, intraperitoneal injection, topical application, or sublingual application.

8. The method of claim 1, wherein the dihydroberberine or a pharmaceutically acceptable salt, acid, ester, polymer, analog or derivative thereof is administered at a daily dose of 10 mg-2000 mg.

9. The method of claim 1, wherein the dihydroberberine or a pharmaceutically acceptable salt, acid, ester, polymer, analog or derivative thereof is formulated in solutions, liquid suspensions, parenteral solutions, injections, tablets, pills, granules, powders, films, (micro)capsules, aerosols, tonics, syrups, beverages, nourishments, snacks, bars, gums, sugars, a facial mask composition, a functionalized cream composition, a functionalized essence, a skin care composition, a make-up composition or a functionalized food composition.

10. The method of claim 1, wherein the method induces BAT thermogenesis or energy expenditure
